(19) United States
(12) Patent Application Publication
(10) Pub. No.: US 2016/0067502 A1
(43) Pub. Date: Mar. 10, 2016

(54) ENCLOSURE FOR PROTECTING A TRIAL NEUROSTIMULATION GENERATOR FROM CONTAMINATION

(71) Applicant: PACESETTER, INC., Sylmar, CA (US)

(72) Inventors: Gene A. Bornzin, Simi Valley, CA (US); Jenner Joseph, Canyon Country, CA (US); Katie Hoberman, Winnetka, CA (US); Zoltan Somogyi, Simi Valley, CA (US); Chris Condit, Plano, TX (US); Heidi Hellman, Los Angeles, CA (US); Armando M. Cappa, Granada Hills, CA (US); Samir Shah, Valencia, CA (US); Geronimo Hernandez, Dallas, TX (US); Federico Gutierrez, Bedford, TX (US); Allan R. Schwartz, Thousand Oaks, CA (US)

(21) Appl. No.: 14/477,623

(22) Filed: Sep. 4, 2014

(51) Int. Cl. A61N 1/375

(52) U.S. Cl. CPC A61N 1/3752

(57) ABSTRACT

Disclosed herein is a disposable enclosure for use with a trial neurostimulation device configured to electrically couple with a neurostimulation lead for implant within a patient. The trial neurostimulation device includes a pulse generator portion. The disposable enclosure includes a first wall structure, a second wall structure opposite the first wall structure, a volume between the first and second wall structures, and a header. The volume is configured to receive therein the pulse generator portion. The header is configured to electrically couple with the neurostimulation lead. The header is supported in the disposable enclosure adjacent the volume and configured to electrically couple with the pulse generator portion when the pulse generator portion is located in the volume.

### FIELD OF THE INVENTION

The disclosure generally relates to trial neurostimulation devices for use with implantable leads. More specifically, the disclosure relates to protective barriers and trial neurostimulation devices configured to inhibit contamination of the trial neurostimulation devices and associated infection transfer.

### BACKGROUND OF THE INVENTION

Implantable neurostimulation devices can be employed to manage pain arising from a variety of neuropathies and is a valuable treatment for chronic intractable neuropathic pain. Neurostimulation is also being investigated for cardiac applications such as treatment of heart failure and atrial fibrillation. To these various ends, a spinal cord stimulation (SCS) device or other neurostimulator may be implanted within the body to deliver electrical pulses to nerves or other tissues. The neurostimulator typically includes a small pulse generator device similar to a pacemaker but equipped to send electrical pulses to leads mounted along the nerves near the spinal cord or elsewhere within the body. For SCS, the generator is often implanted in the abdomen. The stimulation leads may include thin wires or paddles for delivering electrical pulses to patient nerve tissues. An external controller, similar to a remote control device, may be provided to allow the patient to control or adjust the neurostimulation. Currently, prior to permanent (i.e. chronic) implant of a neurostimulator, the patient undergoes a trial period during which he or she is implanted with a percutaneous lead that is externalized and connected to a trial neurostimulation control device or instrument, which the patient carries with him or her.

In United States, patients typically have the trial neurostimulation system for less than a week. In Europe, the trial period can last up to a month. During the trial period, the patient carries the neurostimulation system with him or her. Unfortunately, current trial neurostimulation devices are problematic. The implanted percutaneous lead can be inadvertently pulled from the epidural space or may migrate from the implant site such that the patient will not receive any therapeutic benefit. This can result in a failed trial. In addition, the current system is quite cumbersome. Typically, the lead is taped to the skin at the exit point. A long extension cord connects the lead to the trial neurostimulator, which is worn on a belt. The extension cord and lead are packaged within a bulky bandage and tape arrangement that is uncomfortable and irritating for the patient. With such devices, the patient is not allowed to shower. The trial experience can often be very unpleasant for patients. It is believed that the “annoyance factor” can lead to a failed trial because the patients become “fed up” with the process. As a result, many patients who might benefit from SCS or other forms of neurostimulation do not receive such devices, or the devices are programmed with inappropriate or ineffective parameters. Moreover, the only feedback typically provided regarding therapy effectiveness and optimal stimulation parameters is the subjective feedback given by the patient based on reported sensations.

New trial neurostimulation devices have been developed to address many of the above-described issues. Such trial neurostimulation devices are expensive and should be reused to reduce costs. However, reusing the trial neurostimulation devices presents the threat of cross contamination. Accordingly, there is a need for improved trial neurostimulation devices and associated devices and methods that reduce the likelihood of cross contamination.

### SUMMARY OF THE INVENTION

Disclosed herein is a disposable enclosure for use with a trial neurostimulation device configured to electrically couple with a neurostimulation lead for implant within a patient. The trial neurostimulation device includes a pulse generator portion. In one embodiment, the disposable enclosure incudes a first wall structure, a second wall structure opposite the first wall structure, a volume between the first and second wall structures, and a header. The volume is configured to receive therein the pulse generator portion. The header is configured to electrically couple with the neurostimulation lead. The header is supported in the disposable enclosure adjacent the volume and configured to electrically couple with the pulse generator portion when the pulse generator portion is located in the volume.

In some embodiments, the first and second wall structures may be part of a bag-like structure. One or more of the wall structures may be formed of a pliable medical grade material. Non-limiting examples of material that may be used to form the wall structures includes silicone rubber, polyethylene, polyurethane, polyurethane silicone rubber copolymer, butyl rubber, vinyl, latex, or polyethylene terephthalate.

In some embodiments, the first and second wall structures may be part of respective portions of a clam-shell configuration of the disposable enclosure.

In some embodiments, the header is supported in the disposable enclosure by being fixedly installed into the disposable enclosure as part of the manufacturing of the disposable enclosure. As a non-limiting example, the header may be molded into material extending from one or both of the wall structures. As another non-limiting example, the header may be interference-fit or snap-fit into the disposable enclosure.

In some embodiments, the disposable enclosure further includes an opening extending into the volume. The opening serves as a passage by which the pulse generator portion is inserted into the volume. Such an opening may include a lid configured to close off the opening.

Depending on the embodiment, the header is a lead connector assembly or includes a lead connector assembly. The lead connector assembly includes a mini HDMI connector. The pulse generator portion electrically couples with the header via the mini HDMI connector. The mini HDMI connector may project into the volume.

Also disclosed herein is a method of inhibiting contamination of a pulse generator portion of a trial neurostimulation device that is configured to electrically couple with a neurostimulation lead for implant within a patient. In one embodiment, the method includes enclosing the pulse generator portion in a volume of a disposable enclosure including a first wall and a second wall opposite the first wall. The volume is located between the first and second walls. The enclosed pulse generator portion is attached to the patient. The neurostimulation lead is implanted in the patient and extends through the enclosure and electrically couples with the trial neurostimulation device.

In one embodiment of the method, the first wall is laid down on and adhered to a skin surface of the patient, the pulse generator portion is placed over the first wall, and the second wall is laid down over both the pulse generator portion and the first wall, the second wall being adhered to the first wall. At least one of the first or second walls may include a waterproof medical adhesive tape. The second wall may cover a smaller area than the first wall. The pulse generator portion may be sealed between the first and second walls.

In one embodiment of the method, the disposable enclosure is an inner disposable enclosure, and the method further includes enclosing the inner disposable enclosure within an outer disposable enclosure. The outer disposable enclosure may include a third wall and a fourth wall opposite the third wall. A volume of the outer disposable enclosure may be defined between the third and fourth walls. The enclosed pulse generator portion may be located in the volume of the outer disposable enclosure. The third wall may be laid down on and adhered to a skin surface of the patient. The pulse generator portion enclosed in the inner disposable enclosure may be placed over the third wall. The fourth wall may be laid down over both the pulse generator portion that is enclosed by the inner disposable enclosure and the third wall. The fourth wall may be adhered to the third wall.

Depending on the embodiment of the method, at least one of the third or fourth walls may include a waterproof medical adhesive tape. The fourth wall may cover a smaller area than the third wall. The pulse generator portion may be sealed between the first and second walls, and the inner disposable enclosure may be sealed between the third and fourth walls. The inner disposable enclosure may include a bag-like structure.

In one embodiment of the method, the attaching of the enclosed pulse generator portion to the patient may include affixing the first wall of the outer disposable enclosure to the patient. The disposable enclosure may include a bag-like structure. At least one of the first wall structure or the second wall structure may be formed of a pliable medical grade material. For example, at least one of the first wall structure or the second wall structure may include at least one of silicone rubber, polyethylene, polyurethane, polyurethane silicone rubber copolymer, butyl rubber, vinyl, latex, or polyethylene terephthalate.

In one embodiment of the method, the first and second wall structures are part of respective portions of a clam-shell configuration of the disposable enclosure.

In one embodiment of the method, the disposable enclosure includes a header that is part of the trial neurostimulation device and is supported in the disposable enclosure adjacent the volume. The header and neurostimulation lead may be electrically coupled together as part of electrically coupling the neurostimulation lead and trial neurostimulation device. The method may further include electrically coupling the header and pulse generator portion by the act of enclosing the pulse generator portion in the volume of the disposable enclosure. The header may include a lead connector assembly including a mini HDMI connector. The pulse generator portion may electrically couple with the header via the mini HDMI connector when the pulse generator portion is enclosed in the volume of the disposable enclosure.

Also disclosed herein is a trial header configured for temporary mechanical and electrical coupling with a trial pulse generator including a first electrical coupling component, the trial pulse generator configured to administer neurostimulation to a patient via an implantable lead including a lead connector end. The trial header includes a lead connector receptacle, and a second electrical coupling component. The lead connector receptacle is configured to electrically couple with the lead connector end. The second electrical coupling component is electrically coupled to the lead connector receptacle and configured to electrically couple with the first electrical coupling component when the trial header is a temporarily mechanically mated with the trial pulse generator via a attachable-detachable coupling arrangement defined at least in part in the trial header.

Also disclosed herein is a method of inhibiting contamination of a pulse generator portion of a trial neurostimulation device that is configured to electrically couple with a neurostimulation lead for implant within a patient. The method includes: electrically coupling a lead connector end of the neurostimulation lead implanted in the patient to a lead connector receptacle of a header portion of the trial neurostimulation device; and electrically coupling a first electrical coupling component of the pulse generator portion to a second electrical coupling component of the header portion, the second electrical coupling component being electrically coupled to the lead connector receptacle.

System and method examples are described in detail below.

### BRIEF DESCRIPTION OF THE DRAWINGS

The above and further features, advantages and benefits of the invention will be apparent upon consideration of the descriptions herein taken in conjunction with the accompanying drawings, in which:

FIG. 1 is a diagrammatic depiction of an exemplary trial medical system SCS having an external trial SCS neurostimulation device adhesively attached to the patient and a lead extending from the device into the patient;

FIG. 2 is a side view of the trial SCS neurostimulation device of FIG. 1 and the lead extending from a header of the device;

FIG. 3 is the same view as FIG. 2, except showing the header decoupled from a can or pulse generator portion of the device;

FIG. 4 is a perspective view of a protective enclosure in which the header is preloaded and the can is being loaded via an opening into a cavity of the enclosure;

FIG. 5 is the same view as FIG. 4, except depicting the entirety of the device residing within the confines of the protective enclosure and the opening into the cavity sealed via a door receiving in the opening, and lead connector ends of the leads extending through the wall of the enclosure to be received in corresponding lead connector end receptacles of the header;

FIG. 6 is the same view as FIG. 2, except the device completely resides within the confines of the enclosure and is in surface contact with the patient skin surface, the lead extending from the header and into the patient via a percutaneous opening in the patient;

FIG. 7 is a cross section through the enclosure and device contained therein in the vicinity of the door and as taken along section line 7-7 in FIG. 6;

FIG. 8 illustrates the trial SCS neurostimulation device of FIG. 1 with the lead extending from the header of the device, the device in readiness for affixing to the patient skin surface via a second embodiment of the enclosure, the lead extending from the header and into the patient via a percutaneous opening in the patient;

FIG. 9 is the same view as FIG. 8, except a bottom layer of the enclosure has been laid down on the patient skin surface between the device and the skin surface;

FIG. 10 is a cross section through the device and bottom layer of the enclosure as taken along section line 10-10 in FIG. 9;

FIG. 11 is the same view as FIG. 9, except a top layer of the enclosure has been laid down on the device and the bottom layer to complete the enclosure;

FIG. 12 is a cross section through the device and enclosure as taken along section line 12-12 of FIG. 11;

FIG. 13 illustrates the trial SCS neurostimulation device of FIG. 1 with the lead extending from the header of the device, the device contained in an inner enclosure and in readiness for affixing to the patient skin surface via an outer enclosure, the lead extending from the header and into the patient via a percutaneous opening in the patient;

FIG. 14 is a cross section through the device and inner enclosure as taken along section line 14-14 in FIG. 13;

FIG. 15 is the same view as FIG. 13, except a bottom layer of the outer enclosure has been laid down on the patient skin surface between the inner enclosure and the skin surface;

FIG. 16 is a cross section through the device, inner enclosure and bottom layer of the outer enclosure as taken along section line 16-16 in FIG. 15;

FIG. 17 is the same view as FIG. 15, except a top layer of the outer enclosure has been laid down on the inner enclosure and the bottom layer of the outer enclosure to complete the outer enclosure;

FIG. 18 is a cross section through the device, inner enclosure and outer enclosure as taken along section line 18-18 of FIG. 17, the inner and outer enclosures forming a combined enclosure;

FIG. 19 is a side view of a connector assembly for receiving and connecting with a lead connector end of a lead;

FIG. 20 is a slotted tube of the connector assembly of FIG. 19;

FIG. 21 is a transverse cross section of the connector assembly as taken along section line 21-21 of FIG. 19;

FIG. 22 is a side view of an exterior surface of an outward half of a clamshell protective enclosure;

FIG. 23 is a side view of an interior surface of the outward half depicted in FIG. 22;

FIG. 24 is a side view of an exterior surface of an inward half of the clamshell protective enclosure, this inward half mating with the outward half of FIG. 22 to form the clamshell protective enclosure;

FIG. 25 is a side view of an interior surface of the inward half depicted in FIG. 24;

FIG. 26 is the same view as FIG. 25, except the connector assembly of FIG. 19 has been placed in the upper region of the interior of the inward half of the clamshell protective enclosure;

FIG. 27 is the same view as FIG. 26, except the outward half of the clamshell protective enclosure has been mated with the inward half to form the entirety of the clamshell protective enclosure;

FIG. 28 is the same view as FIG. 27, except lead connector ends are received in the connector assembly and the trial pulse generator portion or can is in the process of being inserted into the clamshell protective enclosure; and

FIG. 29 is the same view as FIG. 28, except the trial can is fully located within the clamshell protective enclosure.

FIGS. 30A-30C are, respectively, isometric, side and exploded side views of trial pulse generator with a disposable header.

FIG. 31 is a diagrammatic representation of a first phase of an intraoperative trial employing the trial pulse generator, wherein a cable connector is employed, the cable connector having a connector assembly that couples with the lead connector ends.

FIG. 32 is a diagrammatic representation of a second phase of an intraoperative trial employing the trial pulse generator and also the trial header disclosed herein, wherein trial header connects with the lead connector ends and a cable connects the trial header to the trial pulse generator.

FIG. 33 is a diagrammatic representation of a first or second phase of a postoperative trial employing the trial pulse generator and also the trial header disclosed herein, wherein trial header connects with the lead connector ends and the trial pulse generator.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators are used to refer to like parts or elements throughout.

### Overview of Trial Neurostimulation System

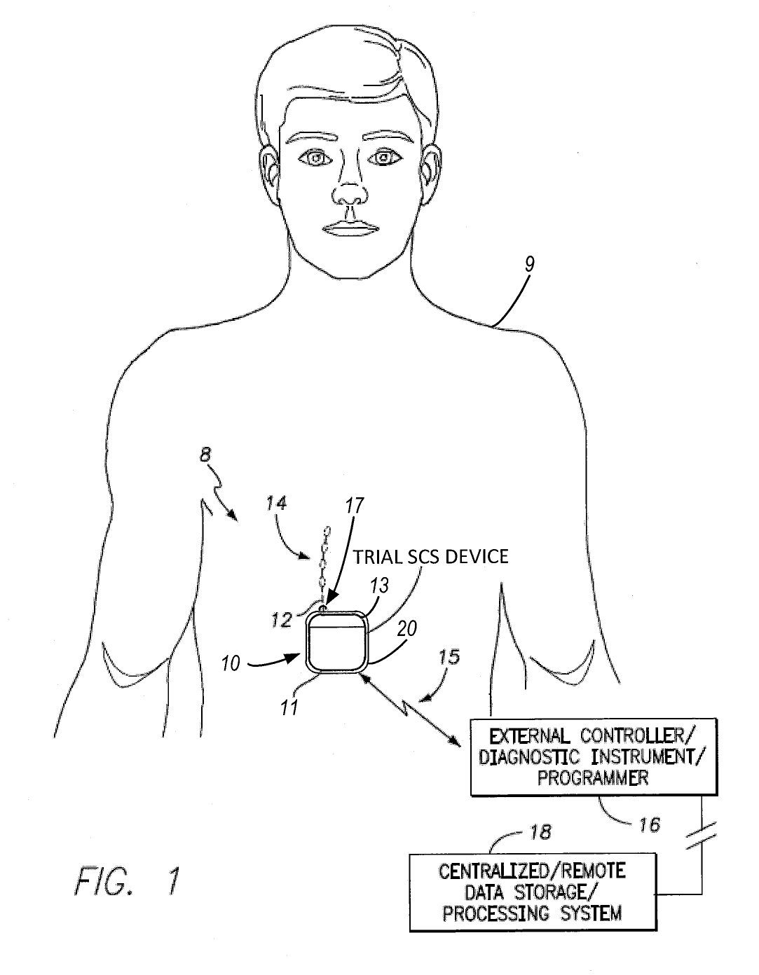

FIG. 1 illustrates an exemplary trial medical system 8 having an external trial SCS neurostimulation device 10 equipped to deliver neurostimulation to a patient 9 on which the device is affixed. The trial SCS device 10 includes a header 13 and a “can” or pulse generator portion 11 of the device 10. The can 11 includes the components of the device 10 that control, sense and/or generate electrical signals that are part of the neurostimulation. The header 13 serves as a coupling structure by which a neurostimulation lead may be coupled to the device 10. The header 13 may be permanently attached to the can 11 or, as discussed below, may be decoupled from can 11 and disposed of.

The trial SCS device 10 employs, in this example, a percutaneous lead 12 with a set of electrodes 14 implanted within the patient 9 for delivering the trial neurostimulation to patient nerve tissues. In the drawing, phantom lines are used to illustrate the implanted portion of the lead 12 whereas solid lines illustrate the external device 10 so as to distinguish the components implanted within the body from those kept external to the patient. The lead 12 enters the patient 9 via a percutaneous puncture or opening 17 in the patient's skin surface.

Although not specifically shown in FIG. 1, a proximal end of the lead 12 is connected into the header portion 13 of the device 10 via an opening in the patient skin so as to allow the pulse generator and other electronics of the SCS device to be externalized from the patient whereas the electrodes 14 along the distal end of the lead are internalized within the tissues of the patient. The percutaneous penetration of the lead 12 into the patient is covered with a bandage and taped down. With this configuration, the point of entry of the lead into the patient can be hygienically sealed.

Typically, the electrodes 14 of a trial SCS lead such as percutaneous lead 12 are positioned near suitable nerves of the spinal column to allow for efficacious pain reduction via neurostimulation. However, in other examples, the electrodes might be placed elsewhere within the patient. Moreover, it should be understood that the percutaneous lead 12 of FIG. 1 is merely exemplary. Four electrodes 14 are shown in the example, although more or fewer electrodes can be employed. For example, the device might employ an eight-electrode Octrode™ lead, which is a type of linear eight electrode percutaneous lead provided by St. Jude Medical. Still further, in other examples, paddle electrode leads or other lead shapes or configurations can be used. Typically, the lead 12 is removed upon completion of the trial period and replaced with a new lead if implantation of a permanent (i.e. chronic or long-term) SCS system is warranted. However, in some examples, the stimulation lead 12 can be retained within the body, with the external device 10 disconnected from the lead and replaced with a fully implantable neurostimulation controller that is then coupled to the implanted lead. See, for example, techniques described in U.S. patent application Ser. No. 13/940,727 of Nabutovsky et al., filed Jul. 12, 2013, entitled “Fully Implantable Trial Neurostimulation System Configured for Minimally-Intrusive Implant/Explant” (Atty. Docket A13P3007.)

In the example of FIG. 1, the trial SCS device 10 is equipped to communicate with an external controller/diagnostic instrument/programmer 16 using radio-frequency (RF) or other wireless signals to transmit data collected by the trial device (including data pertaining to patient pain) and/or to receive commands from the external instrument to activate, deactivate or adjust neurostimulation. The commands may specify various stimulation sets (Stim Sets) initially specified by a clinician. The Stim Sets specify SCS parameters for controlling delivery of SCS to nerve tissues of the patient to address the needs of the patient, such as to reduce pain or to achieve desired cardioprotective effects. The clinician or the patient can then change the Stim Sets using external the instrument 16 via a wireless communication link 15 such as to change the amplitude, frequency or duration of stimulation pulses generated by the SCS device. The communication link may employ Bluetooth or other suitable wireless communication protocols. In some examples, the external instrument 16 is a suitably-equipped tablet computer or smartphone, which may be referred to as a “Neuro External” device. See, for example, U.S. patent application Ser. No. 14/012,634 of Wu et al., filed Aug. 28, 2013, entitled “Systems and Methods for Low Energy Wake-Up and Pairing for use with Implantable Medical Devices” (Atty Docket A13P3012). The external instrument 16 may also be equipped to communicate with a centralized/remote data processing system 18 via the Internet or other suitable communication channels/networks to relay information to the primary care physician of the patient or to other appropriate clinicians. The centralized system may include or employ such systems as the HouseCall™ remote monitoring system or the Merlin@home/Merlin.Net systems of St. Jude Medical.

Although the example of FIG. 1 shows a trial device 10 for stimulating the spinal cord, additional or alternative stimulation devices might be employed, such as devices for stimulating other tissues or organs within the patient. Some patients might additionally have an implantable cardiac rhythm management device (CRMD) such as a pacemaker, implantable cardioverter-defibrillator (ICD) or a cardiac resynchronization therapy device (CRT), which is not shown in the figure. Note also that FIG. 1 is a stylized illustration that does not necessarily set forth the precise locations of the various device components nor their relative sizes or shapes.

Disposable Header for Use with Trial SVC Devices

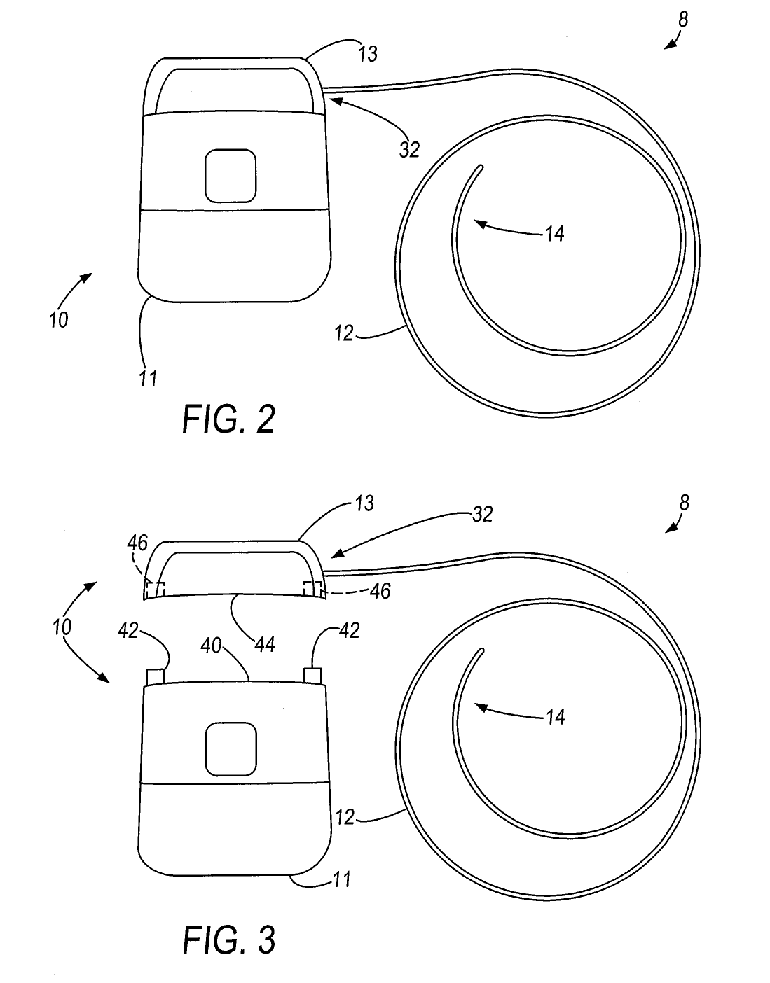

FIGS. 2 and 3 illustrate details of an exemplary trial SCS device 10 that does not have a bulky extension, is meant to be taped directly to the patient and, further, has a header 13 configured to be coupled to and decoupled from the external pulse generator or can 11. The proximal end of the lead 12 plugs directly into header 13 on the trial SCS device 10.

As depicted in FIG. 2, when the trial SCS device 10 is to be employed during a period of trial neurostimulation, the external pulse generator or can 11 is coupled to the header 13 and both are affixed as the whole device 10 to the patient. The proximal end of the lead 12 is plugged into an appropriate lead connector receptacle 32 in the header 13, and electrical circuits extending through the header 13 place the lead circuitry into electrical communication with the electrical circuitry of the can 11, which is coupled both mechanically and electrically to the header 13. As illustrated in FIG. 3, when the trial is complete or the trial SCS device 10 is otherwise to be removed from the patient, the header 13 can be decoupled from the external pulse generator or can 11 with the proximal end of lead 12 still coupled to header 13.

The lead 12, including its proximal end, becomes contaminated with body fluids during implantation of the lead into the patient. Because the entirety of the lead is contaminated, the header 13 will also become contaminated as a function of the lead proximal end being received in a lead connector receptacle 32 of the header 13 during the course of the lead proximal end being coupled to the header 13. In addition to being configured to decouple from the can 11 of the trial SCS device 10, in some embodiments the header 13 is further designed to be disposable, thereby avoiding having to process for reuse the header 13. The ability of the header 13 to decouple from the can 11 and to be disposed of reduces the probability of infection transfer and saves time and money, as reprocessing expenses and time can be limited to the reprocessing of the can 11 of the trial SCS device 10, the can 11 being too expensive to be disposable.

In other embodiments, the header 13 can be configured for sterilization in an autoclave or via other sterilization methods.

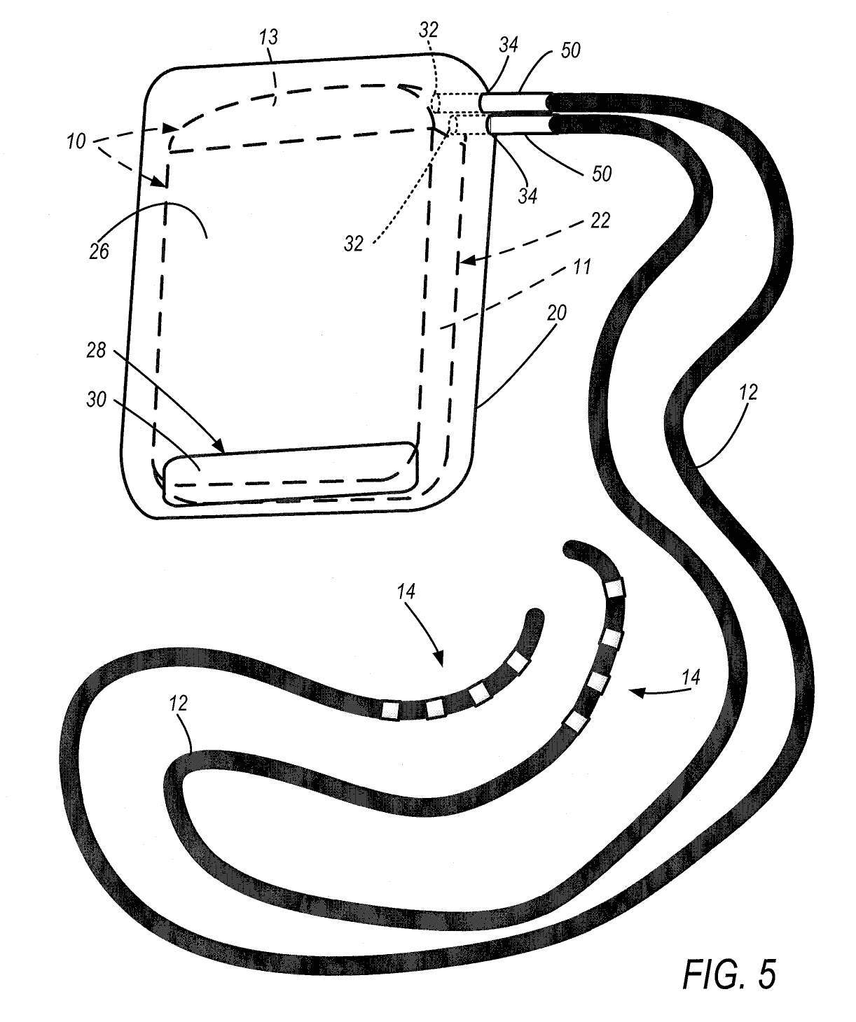

As can be understood from FIGS. 2 and 3, the can 11 of the device 10 includes a header-interface surface 40 with posts 42 or other male structures projecting outwardly from the surface 40. The header 13 includes a can-interface surface 44 with female features 46 that are complementary to the posts 42 to receive the male posts 42 when the two surfaces 40, 44 abut in mating contact as indicated in FIGS. 2 and 5. With the two surfaces 40, 44 so abutted and the posts 42 received in the female counterparts 46 in the surface 44, the header 13 and can 11 are secured together and their respective electrical components are electrically connected so as to allow the device 10 to be electrically complete as a whole.

An alternative embodiment of the device is also depicted in FIGS. 30A-30C, wherein a male post 42 extends from the can-interface surface 44 of the header 13 to be received in the complementary female feature 46 of the header-interface surface 40 of the can 11. The header 13 is shown to have two lead connector receptacles 32. Of course, the header 13 can be configured to have any number of lead connector receptacles 32 as required for the type of neuromodulation intended to be administered. The lead connector receptacles 32 can also be electrically and mechanically coupled with the lead connector ends of any type of lead, including, for example, octrode and quatrode type leads, which each have multiple electrodes and corresponding electrical connections requiring electrical connection to the pulse generator's circuit. The lead connector receptacles 32 may be configured like implantable pulse generators or may take a different mechanical form factor. The header can be configured to establish electrical connections to the body of the pulse generator providing the trial stimulation therapy.

The header 13 and its receptacles 32 may be configured to allow a stylet or guide wire to be in place in the lead 12 when the lead is being inserted into header. Further, the receptacles 32 may be configured so as to ensure that lead insertion and removal requires an acceptable and usable force. The header 13 may be configured to allow an additional lead to be inserted into the header without affecting the position of leads already in the header. Finally, the receptacles 32 hold and retain the lead connector ends of the leads in the header through forces expected to be exerted on the lead during intra-operative testing or trial, thereby ensuring that electrical contact between the lead, header and can is maintained while the system is subjected to movement or vibration while worn by a patient.

The header and can interfaces may be configured to guide the header into a coupled arrangement with the pulse generator such that the circuit coupling features 42, 46 are fully mechanically and electrically coupled together and the interfaces 40, 44 are fully mechanically coupled together such that they hold and retain the header onto the pulse generator in such a way to endure forces expected to occur during a trial, such as a patient lying on the connected pulse generator and header.

The header 13 may be fully encased to make it robust, resistant to fluid intrusion, and reliable. The header may be disposable or configured such that it can survive sterilization processes.

In addition to inhibiting infection transfer and reducing associate costs, the trial SCS device 10 of FIGS. 1-3 has several other advantages. First, the device 10 is smaller than present belt worn generators and can be taped to the patient's skin. Second, the device 10 “out of sight” under the patient's clothing. Finally, the lead 12 plugs directly into a disposable header 13, thereby eliminating the need for an extension. These three advantages improve patient comfort and the ability of the patient to discretely employ the device 10.

The above-describe header 13 and can 11 of the trial SCS device 10 of FIGS. 2 and 3 provide substantial improvement in patient care, comfort and outcome. Also, the header 13 and can 11 of the device 10 reduce the likelihood of infection via the lead percutaneous access and reduce the likelihood of infection transfer between patients employing the reusable can 11 of the trial SCS device 10.

Regardless of whether the header 13 illustrated in FIGS. 2, 3 and 30A-30C is capable of being sterilized and reused or is a single-use header 13, the header 13 connects leads 12 to an external pulse generator 11 in a style that mimics headers on implantable pulse generators. This has the advantage of eliminating the intervening uncomfortable cable connector currently used to connect the leads to the external pulse generator.

As already stated above, the leads 12 become contaminated with body fluids, but must connect to the external pulse generator 11. In order to reduce the probability of infection transfer and to avoid the step of processing or re-sterilizing the external pulse generator 11 between trial patients, the header 13 disclosed herein connects directly to the external pulse generator 11 and also directly to the leads 12.

The header 13 may be configured for sterilization and reuse. Alternatively, the head 13 may be a single-use disposable component. Such a header 13 is inexpensive so that using one per patient is not cost prohibitive. The entire header 13 snaps on to the external pulse generator 11 and can be worn and bandaged comfortably. In some embodiments, despite being inexpensive enough to be disposable, the header 13 can additionally be configured to survive sterilization so that it could be introduced into the sterile field if desired.

As can be understood from FIGS. 1, 30A-30C and 33, in one embodiment, the trial header 13 is configured for temporary mechanical and electrical coupling with the trial pulse generator 11 to form the trial pulse generator system 10. The trial pulse generator 11 includes a first electrical coupling component 46. The trial pulse generator 11 is configured to administer neurostimulation to the patient 9 via the implanted electrodes 14 of one or more implantable leads 12. Each lead includes a lead connector end 50

The trial header 13 includes one or more lead connector receptacles 32 and a second electrical coupling component 42. The one or more lead connector receptacles 32 are configured to electrically couple with the lead connector ends 50. The second electrical coupling component 42 electrically is coupled to the lead connector receptacles 32 and configured to electrically couple with the first electrical coupling component 46 when the trial header 13 is a temporarily mechanically mated with the trial pulse generator 11 via a attachable-detachable coupling arrangement defined at least in part in or between the respective complementary interfacing surfaces 44, 40 of the trial header 13 and trial pulse generator 11.

For example, in one embodiment, the attachable-detachable coupling arrangement is an interference-fit configuration between the trial pulse generator and the trial header or, more specifically, between the contours or features of the interfacing surfaces 40, 44. In other words, in one embodiment, the attachable-detachable coupling arrangement may include a contoured interface surface 44 of the trial header 13 that structurally mates with a complementary contoured interface surface 40 of the trial pulse generator 11.

Additionally, in one embodiment as can be understood from FIG. 30C, the attachable-detachable coupling arrangement may further include a first structural feature surrounding the first electrical coupling component 46 and a second structural feature surrounding the second electrical coupling component 42, these first and second structural features being configured to mechanically couple with each other. Such a mechanical coupling may be on account of the male-female coupling arrangement depicted in FIG. 30C, wherein the second electrical coupling component 42 is a male half of an electrical plug (e.g., a male HDMI connector or even a male mini-HDMI connector), and the first electrical coupling component 42 is a female half of the electrical plug (e.g., a female HDMI connector or even a female mini-HDMI connector).

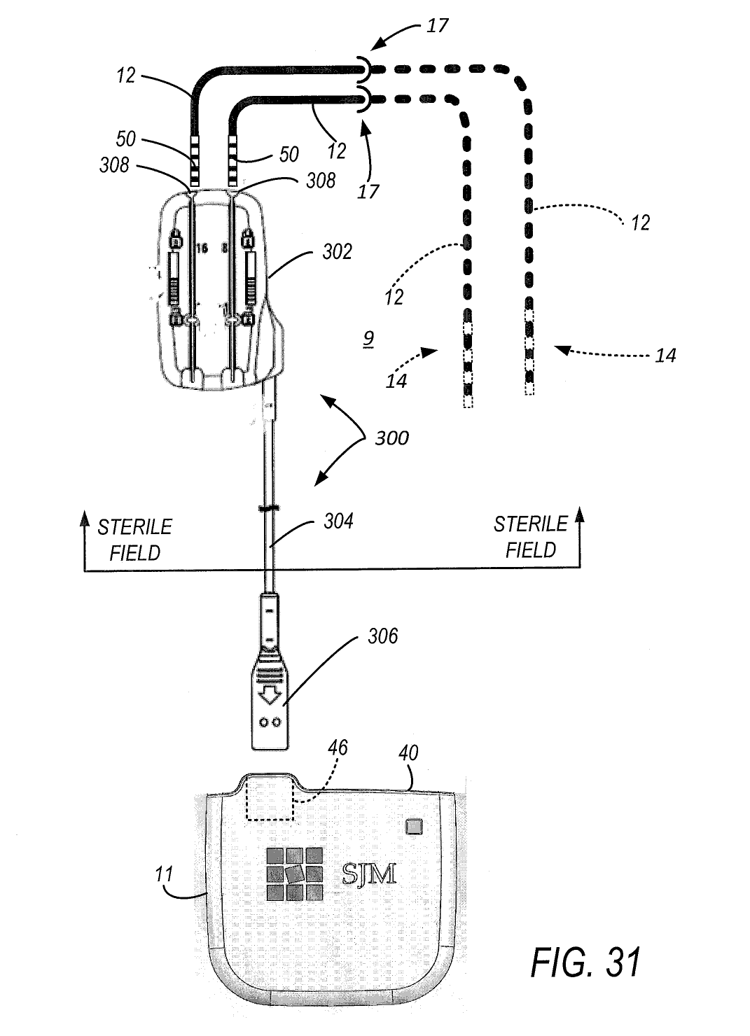

FIG. 31 is a diagrammatic representation of a first phase of an intraoperative trial employing the trial or exterior pulse generator 11. As shown in FIG. 31, a cable connector 300 couples the leads 12 to the trial or exterior pulse generator 11. The lead bodies 12 extend distally from their respective lead connector ends 50 into the patient 9 via percutaneous openings 17, the rest of the lead bodies 12 distal the openings 17 being implanted in the patient 9 such that the electrodes 14 supported on the lead bodies 12 are implanted in the patient 9.

The cable connector 300 includes a connector assembly 302 and a cable 304 that extends from the assembly 302 to proximally terminate in the form of a plug 306 that can be plugged into the female plug receptacle 46 in the pulse generator 11. The plug 306 may be in the form of an HDMI connector or even a mini-HDMI connector. The connector assembly 302 includes receptacles 308 that are configured to receive therein, and electrically and mechanically coupled with, the lead connector ends 50 to place the lead circuits extending from the electrodes 14 in electrical communication with the cable 304 and the plug 306.

As can be understood from FIG. 31, with the lead connector ends 50 received in the receptacles 308, the plug 306 can be received in the receptacle 46 of the pulse generator 11 such that the cable connector 300 places the electrodes 14 in electrical communication with the electrical circuits of the trial pulse generator 11. The leads 12 and the connector assembly 302 of the cable connector 300 are located in the sterile field. The cable 304 extends from the sterile field into the non-sterile field such that the plug 306 and the trial pulse generator 11 are located in the non-sterile field.

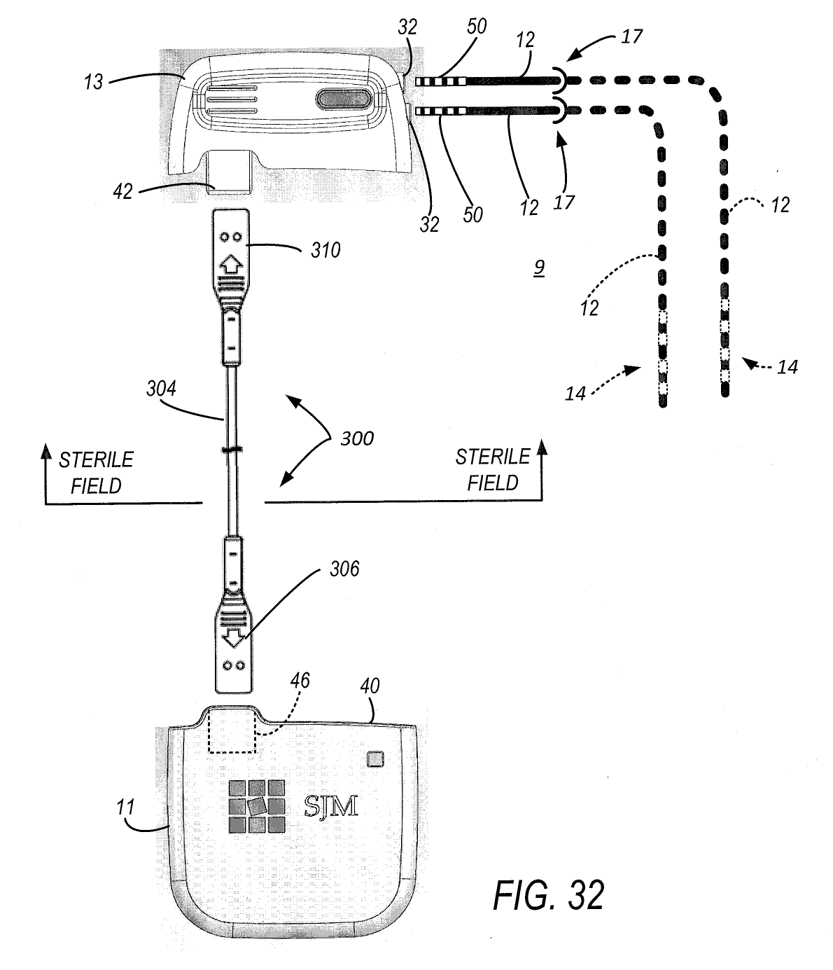

FIG. 32 is a diagrammatic representation of a second phase of an intraoperative trial employing the trial or exterior pulse generator 11. As shown in FIG. 32, a trial header 13 and a cable connector 300 work together to couple the leads 12 to the trial or exterior pulse generator 11. The lead bodies 12 extend distally from their respective lead connector ends 50 into the patient 9 via percutaneous openings 17, the rest of the lead bodies 12 distal the openings 17 being implanted in the patient 9 such that the electrodes 14 supported on the lead bodies 12 are implanted in the patient 9.

The cable connector 300 includes a cable 304 that extends between a proximal plug 306 and a distal plug 310. The distal plug 310 is electrically coupled with the plug receptacle 42 of the trial header 13 discussed above. Similarly, the proximal plug 306 is electrically coupled with the plug receptacle 46 in the pulse generator 11. The one or more of the plugs 306, 310 may be in the form of an HDMI connector or even a mini-HDMI connector. The trial header 13 includes receptacles 32 that are configured to receive therein, and electrically and mechanically couple with, the lead connector ends 50 to place the lead circuits extending from the electrodes 14 in electrical communication with the electrical circuits of the trial header 13.

As can be understood from FIG. 32, with the lead connector ends 50 received in the receptacles 32 and the distal plug 310 electrically coupled to the electrical circuits of the header 13, the proximal plug 306 can be received in the receptacle 46 of the pulse generator 11 such that the cable connector 300 places the electrodes 14 in electrical communication with the electrical circuits of the trial pulse generator 11. The leads 12, trial header 13 and the distal plug 310 of the cable connector 300 are located in the sterile field. The cable 304 extends from the sterile field into the non-sterile field such that the proximal plug 306 and the trial pulse generator 11 are located in the non-sterile field.

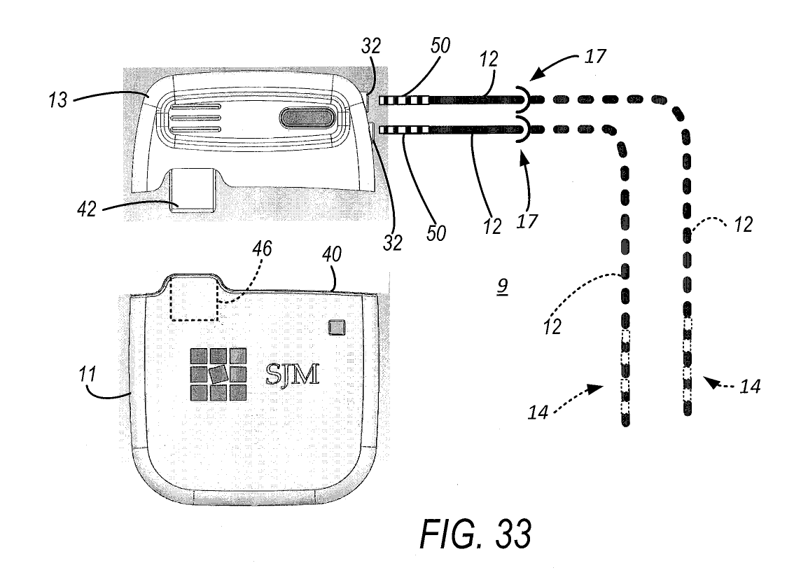

FIG. 33 is a diagrammatic representation of a first or second phase of a postoperative trial employing the trial or exterior pulse generator 11. As shown in FIG. 33, the trial header 13 couples the leads 12 to the trial or exterior pulse generator 11 without the use of a cable connector 300. The lead bodies 12 extend distally from their respective lead connector ends 50 into the patient 9 via percutaneous openings 17, the rest of the lead bodies 12 distal the openings 17 being implanted in the patient 9 such that the electrodes 14 supported on the lead bodies 12 are implanted in the patient 9.

The trial header 13 includes receptacles 32 that are configured to receive therein, and electrically and mechanically couple with, the lead connector ends 50 to place the lead circuits extending from the electrodes 14 in electrical communication with the electrical circuits of the trial header 13.

As can be understood from FIG. 33, with the lead connector ends 50 received in the receptacles 32, the header plug 42 is electrically and mechanically coupled to the receptacle 46 of the trial pulse generator 11 such that the electrodes 14 are in electrical communication with the electrical circuits of the trial pulse generator 11. The boundary of the sterile field can be said to extend across the trial header 13 such that any contamination associated with the leads 12 does not extend across the trial header 13 to the trial pulse generator 11 to which the trial header 13 is connected.

Further improvements regarding patient comfort and infection inhibition can be provided by employing the above-described header 13 and can 11 of the trial SCS device 10 with any of the protective enclosures 20 that will be discussed below. These protective enclosures 20 will facilitate the patient showering with caution. Also, because the “tape on” and “bandaging” processes are still challenging and inconsistent from one clinical practitioner to another, it is possible, although unlikely, that the can 11 of the trial SCS device 10 may become contaminated with infectious matter when it is taped in place directly on the patient's skin. Although unlikely, this contamination may be passed on to another patient. Accordingly, employing any of the below-discussed protective enclosures 20 with the above-described can 11 and disposable header 13 of the device 10 will make the likelihood of infection transfer between patients even more unlikely.

### Protective Enclosures for Inhibiting Contamination of Trial SVC Devices

An additional component for the above-describe trial SCS device 10 is a protective enclosure system 20 for the device 10. The enclosure system 20 provides a water barrier and medical seal around the trial SCS device 10. Accordingly, the enclosure 20 keeps water away from the device 10 during showering and also prevents bacteria, fungi, and viruses from contaminating the surface of the device 10 and thus transferring the contamination from patient to patient. In some embodiments, the enclosure 20 may also provide a connector interface between the lead proximal end and the can 11 of the device 10. The enclosure 20 may be made of many different materials and in many different configurations.

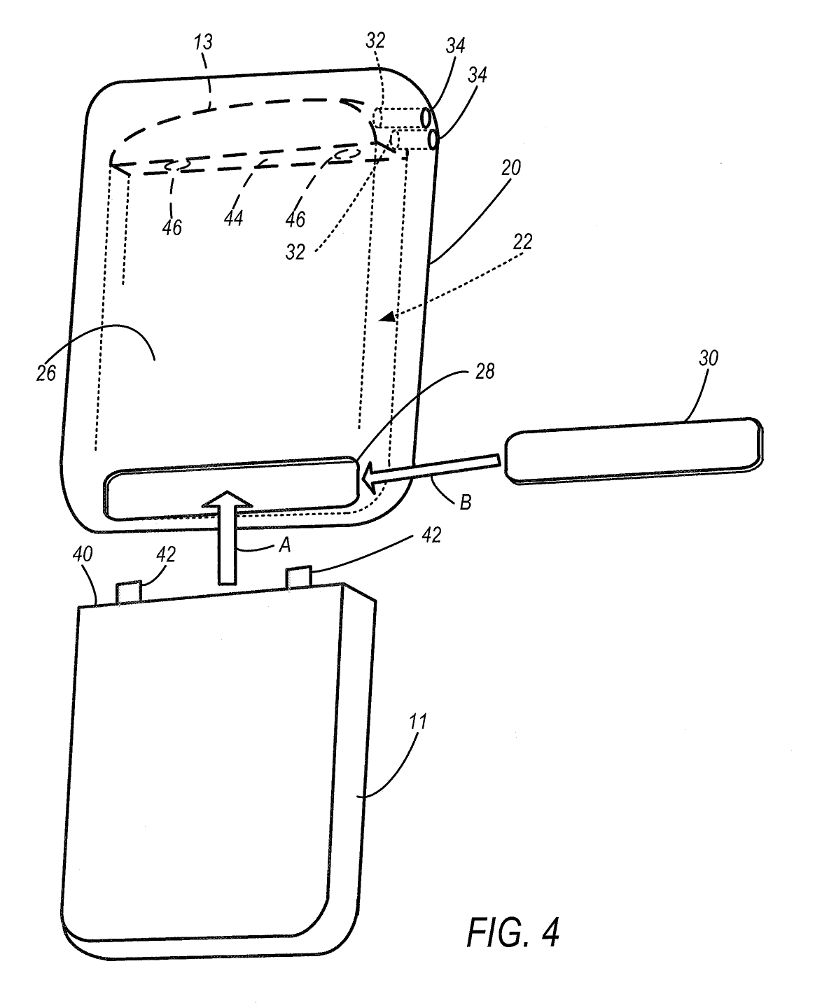

FIG. 4 illustrates one embodiment of an enclosure 20 wherein the enclosure is a bag-like structure configured to receive therein and protect the can 11 of the trial SCS device 10. The enclosure 20 may be molded of soft flexible silicone rubber, polyethylene, polyurethane, polyurethane silicone rubber copolymer, butyl rubber, vinyl, latex, polyethylene terephthalate (“PET”) or similar pliable medical grade materials. The enclosure 20 includes an interior void 22 that has a shape and volume that substantially mimics the shape and volume of the can 11 to be received in the interior cavity 22. The enclosure 20 includes a patient side 24 (shown in FIG. 7) and an outward-facing side 26 opposite the patient side 24. The patient side 24 is substantially planar and configured to making abutting surface contact with the patient's skin surface. The patient side 24 may include a sticky medical adhesive to help affix the enclosure 20, and the trial SCS device 10 enclosed therein, in abutting surface contact with the patient's skin surface. Alternatively or additionally, the enclosure 20 may be taped to the patient using water-resistant medical tape.

The enclosure 20 also includes an access opening 28 leading into the interior cavity 22. As indicated by Arrow A in FIG. 4, the can 11 may be loaded into the cavity 22 via being fully inserted through the access opening 28 so the can 11 occupies the cavity 22 as depicted in FIGS. 5-7. As indicated by Arrow B in FIG. 4 and as can be understood from FIGS. 5-7, the enclosure 20 also includes a lid or door 30 configured to be received in the opening 28 to occupy and close the opening 28, thereby sealing can 11 within the cavity 22. In one embodiment, the door 30 snap-fits into the opening 28, which is located at a bottom region of the enclosure 20 opposite the upper region of the enclosure occupied by the header 13.

As can be understood from FIG. 4, the above-described disposable header 13 is preloaded into the top of the cavity 22 prior to the delivery of the can 11 into the cavity 22. The preloading of the header 13 into the cavity 22 may be achieved by simply passing the header 13 through the opening and into the upper region of the cavity 22 to be positioned as indicated in FIG. 4. Alternatively, the header 13 may be molded into the enclosure 20 such that the header 13 is positioned within the enclosure 20 as depicted in FIG. 4. Regardless, of how the header 13 ends up positioned within the enclosure 20 as depicted in FIG. 4, the lead connector receptacles 32 of the header 13 will be aligned with corresponding openings 34 extending through the wall 36 (shown in FIG. 7) of the enclosure 20.

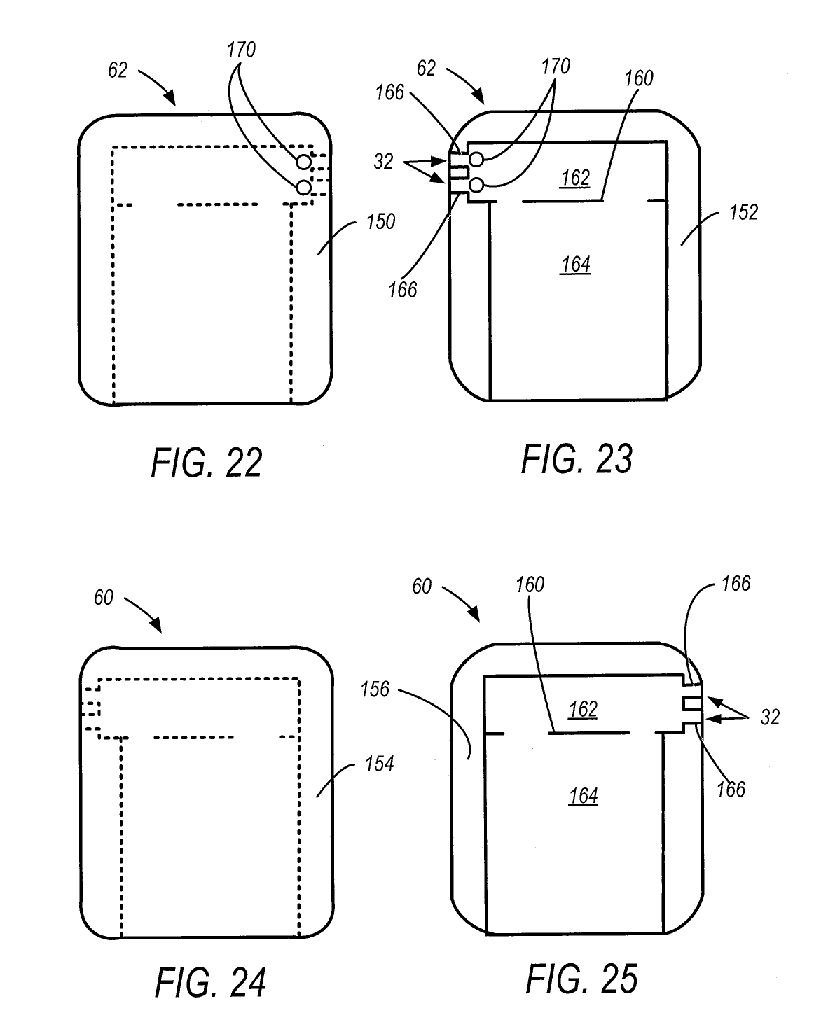

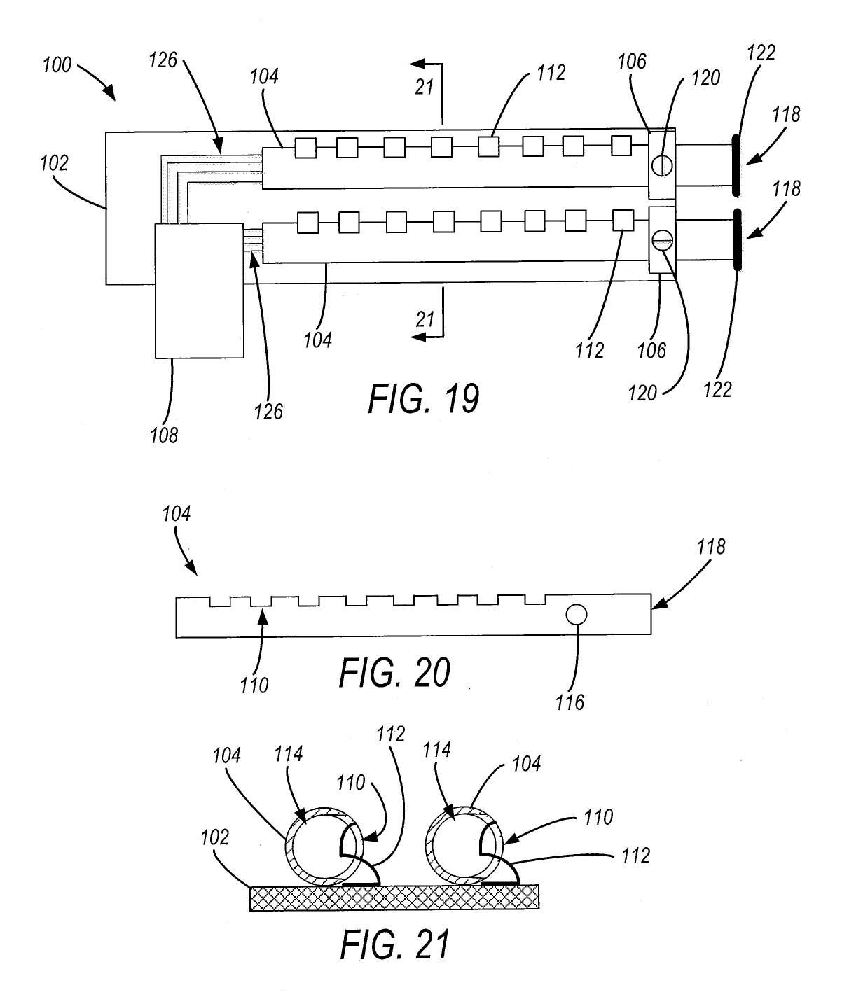

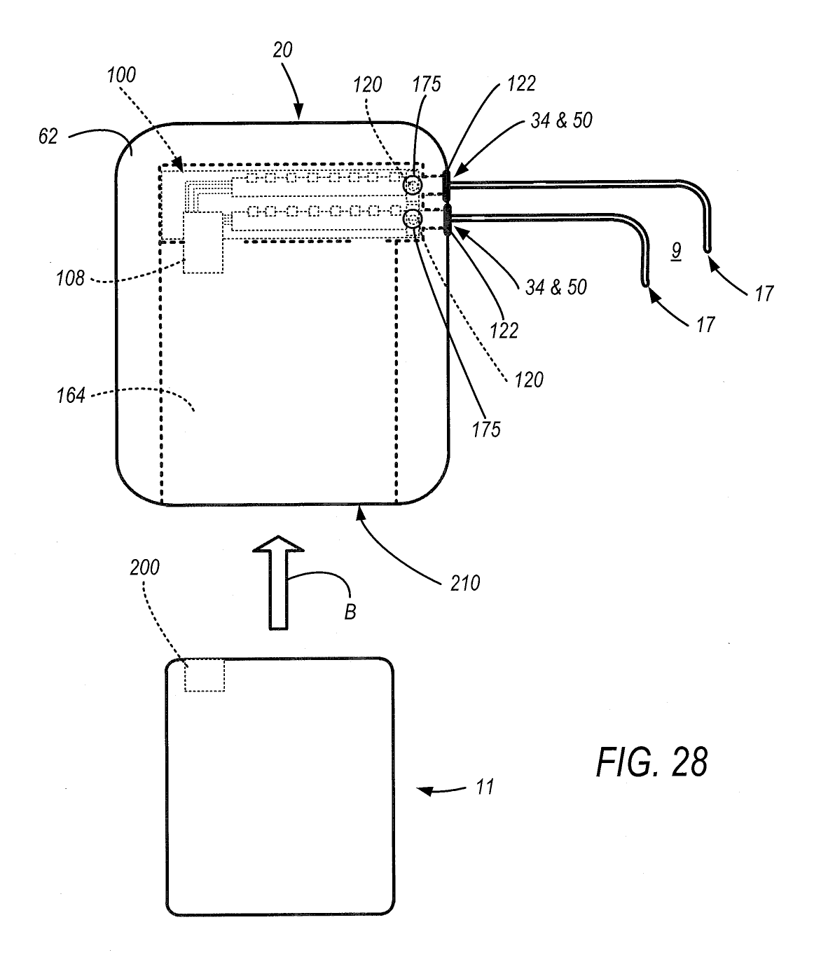

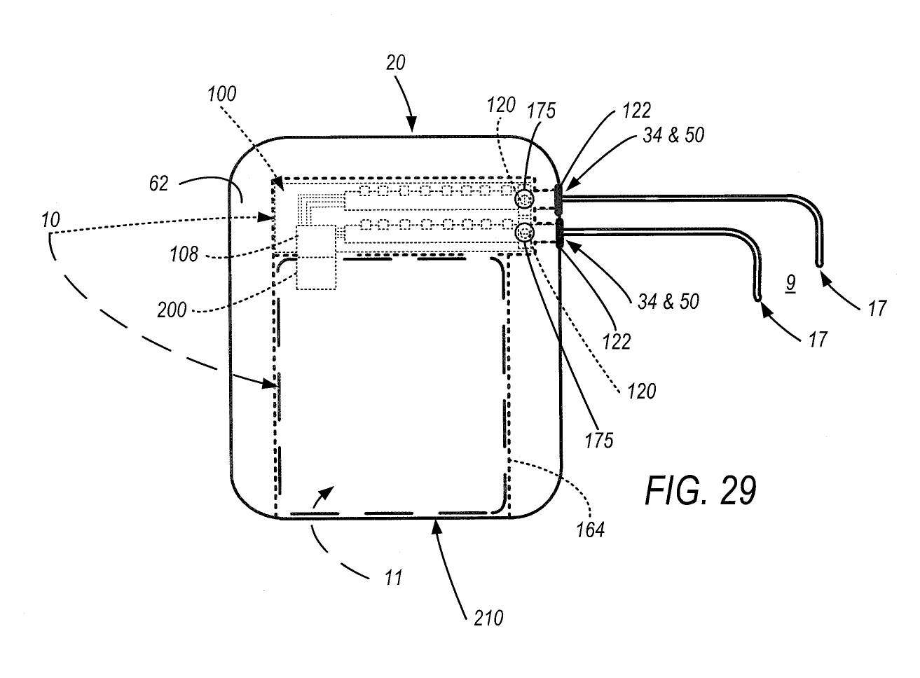

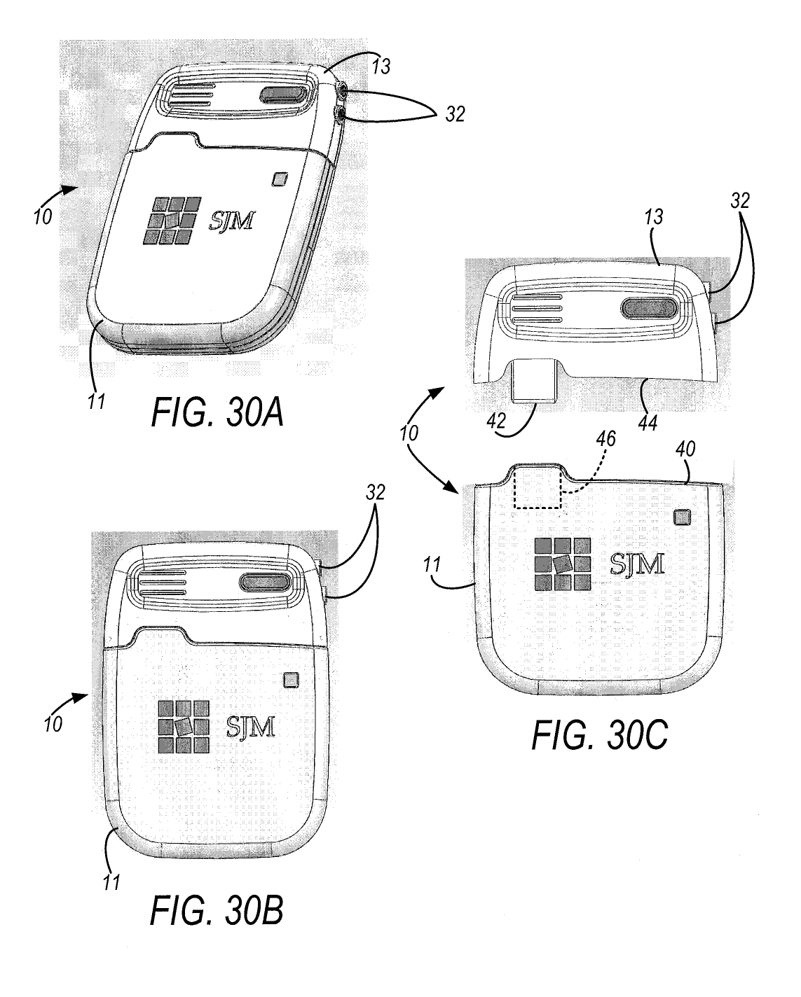

As illustrated in FIGS. 3 and 4, the can 11 of the device 10 includes a header-interface surface 40 with posts 42 or other structures projecting outwardly from the surface 40. The header 13 includes a can-interface surface 44 with female features 46 that are complementary to the posts 42 to receive the posts 42 when the two surfaces 40, 44 abut in mating contact as indicated in FIGS. 2 and 5. With the two surfaces 40, 44 so abutted and the posts 42 received in the female counterparts 46 in the surface 44, the header 13 and can 11 are secured together and their respective electrical components are electrically connected so as to allow the device 10 to be electrically complete as a whole. Alternatively, the header 13 may be or include the lead connector assembly 100 described below with respect to FIG. 19. The lead connector assembly 100 may include a mini HDMI connector 108 and the can or pulse generator portion 11 may be configured to electrically couple with the header 13 via the mini HDMI connector 108 as discussed below with respect to FIGS. 28 and 29.

As illustrated in FIG. 5, once the device 10 is fully assembled and complete within the cavity 22 of the enclosure 20, the lead connector ends 50 at the proximal end of the leads 12 may be inserted through the wall openings 34 and into the corresponding lead connector receptacles 32 of the header 13, thereby placing each lead 12 into electrical communication with the appropriate circuits within the can 11. A tight water seal between the wall openings 34 and the lead connector ends 50 extending there through prevents water and microorganisms from penetrating to the header 13 and the electronics of the can 11

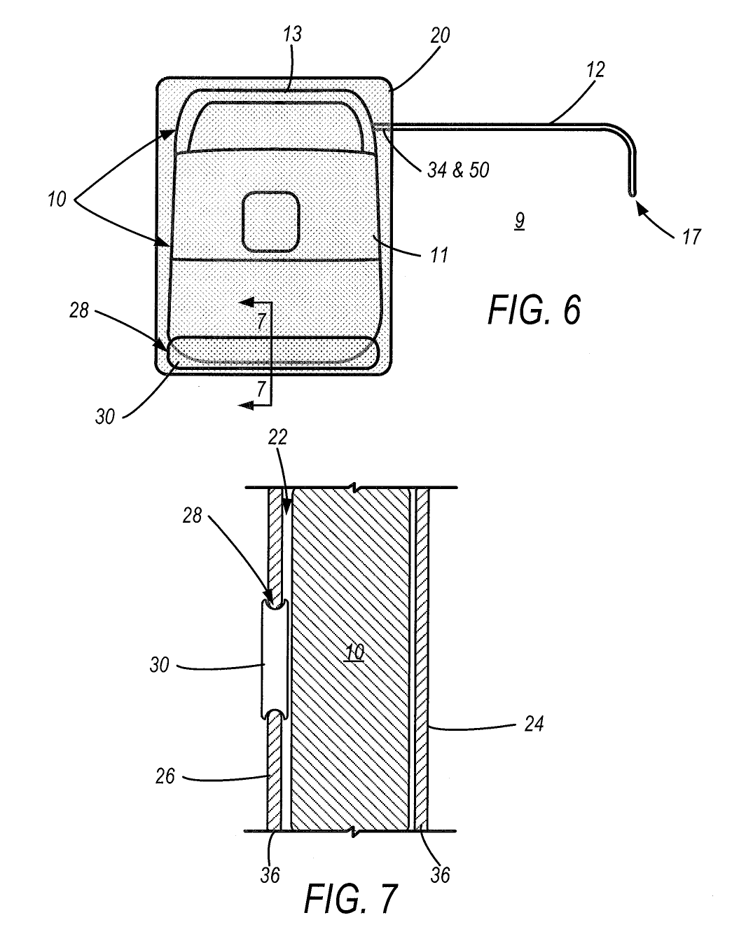

As can be understood from FIGS. 1 and 6, the lead 12 extends from the device header 13 into the patient 9 via the percutaneous entry 17. The lead 12 is taped down around the entry 17 to secure the lead to the patient and to seal the entry 17. The enclosure 20, with the device 10 fully contained within the enclosure 20, is affixed to the patient 9 via an adhesive back surface 24 of the enclosure or via other methods such as, for example, taping the enclosure 20 to the patient 9.

In use, all of the elements of FIG. 4 may be initially handled by a medical professional (e.g., the surgeon) in the sterile field. For example, in the sterile field the can 11 is inserted into and sealed within the enclosure 20. The loaded enclosure 20 of FIGS. 5 and 6 is affixed to the patient for the trial period. Following the trial period or when the can 11 otherwise requires attention, the door 30 is removed from the opening 28 and the can 11 is removed from the cavity 22 via the opening 28. New batteries may be installed into the can 11 and the can 11 may be inserted into another enclosure 20 and reused for the next patient. It should be noted that at no time does the can 11 come in contact with the patient's skin and the can 11 does not get contaminated with the body fluids that contaminate the lead 12.

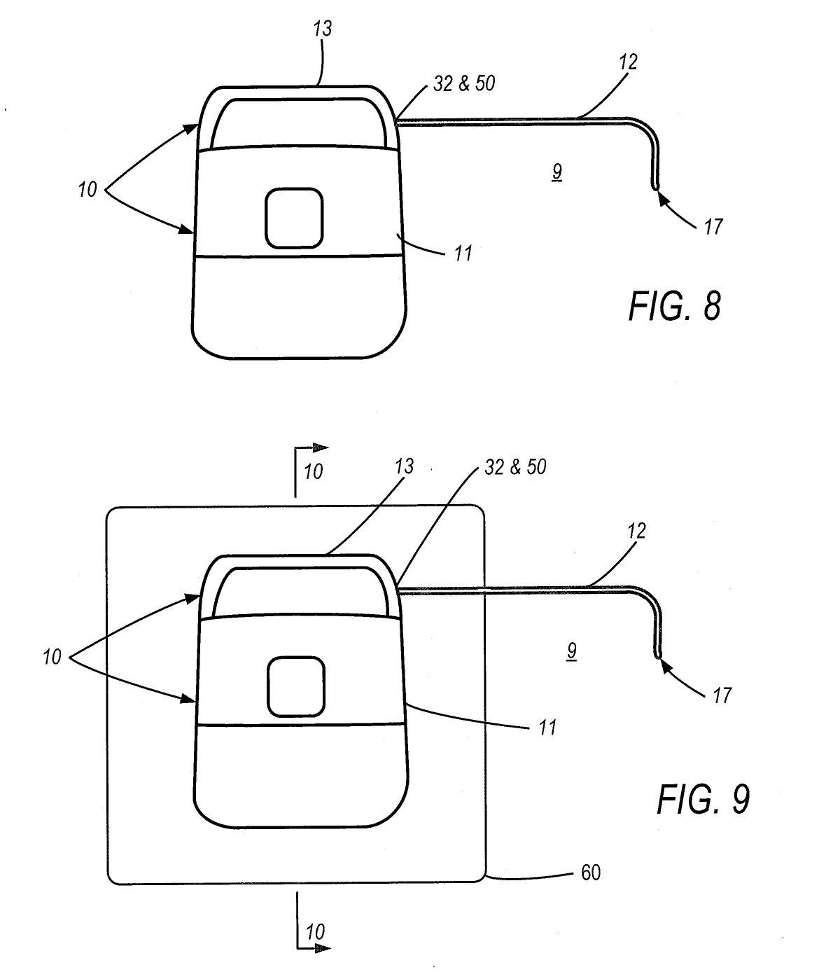

FIG. 8 illustrates the trial SCS neurostimulation device 10 of FIG. 1 in readiness for affixing to the patient skin 9 surface via a second embodiment of the enclosure 20. Specifically, the lead 12, which has been implanted in the patient 9, extends from the header 13 of the device 10 and into the patient 9 via a percutaneous opening 17 in the patient 9.

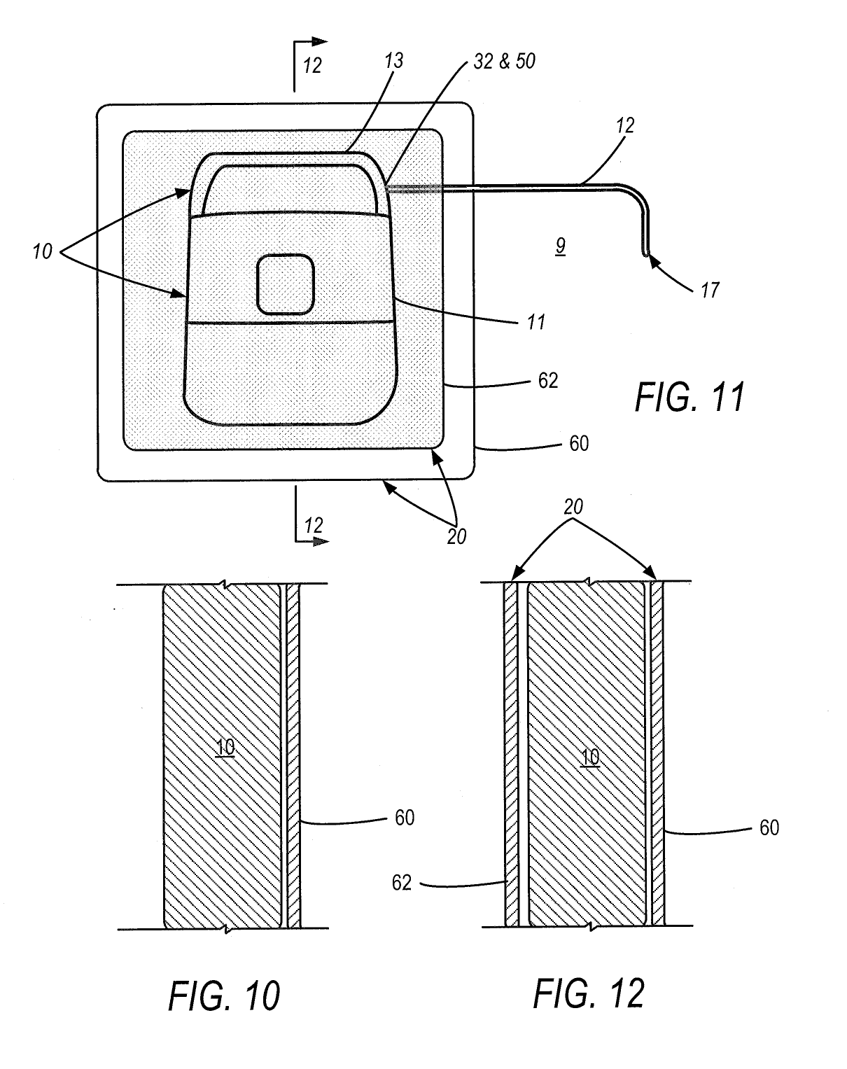

As shown in FIGS. 9 and 10, a bottom layer 60 of the enclosure 20 has been laid down on the patient skin surface 9 between the device 10 and the skin surface 9. The bottom layer 60 may be a waterproof medical adhesive tape 60 or other waterproof material that is adhered to the patient skin surface. The tape 60 not only tolerates the presence of water but does not allow water to diffuse through the tape 60. The bottom layer 60 is sufficiently larger in area than the device 10 to isolate the device 10 from the patient skin surface 9.

As illustrated in FIGS. 11 and 12, the enclosure 20 is completed by applying a top layer 62 of the enclosure 20 over the device 10 and the bottom layer 60. The top layer 62 may be a waterproof medical adhesive tape 62 or other waterproof material that is adhered to the device 10 and the bottom layer 60 to sandwich the device 10 between the two layers 60, 62 to fully contain the device 10 in a sealed enclosure 20. The top layer 62 is sufficiently larger in area than the device 10 but, in some embodiments, may be smaller in area than the bottom layer 60. By being sealed between the two layers 60, 62 of the enclosure 20, the waterproof medical tape prevents water from the patient showering from reaching the device 10 and also isolates the device 10 from contamination via other means. The two tape layers 60, 62 also seal around the lead 12 adequately to keep the connector 50 and device 10 isolated from the surrounding environment, including water and contaminants.

Following use, the bottom layer 60 is peeled off the patient 9 along with the trial SCS device 10 and the rest of the enclosure 20. Then the two layers 60, 62 of the enclosure 20 are removed from about the device 10. Finally, the header 13 is discarded because it is contaminated with body fluids that end up on the lead 13 due to the lead implantation process. New batteries are inserted in the can 11 of the device 10 and the can 11 may be reused for the next patient.

It should be noted that the enclosure 20 of FIGS. 11 and 12 prevents the can 11 of the device 10 from coming into contact with the patient skin surface 9. Further, enclosure 20 prevents the can 11 from getting contaminated with the body fluids that contaminate the lead 12.

In one embodiment, one or both layers 60, 62 of the enclosure 20 of FIGS. 11 and 12 are made from a micro-porous tape made of a hydrophobic material. Such a material can pass water vapor and thus provide a more comfortable breathable barrier as long as the pores are in the micron range to prevent liquid water or contaminants from passing through the layers 60, 62.

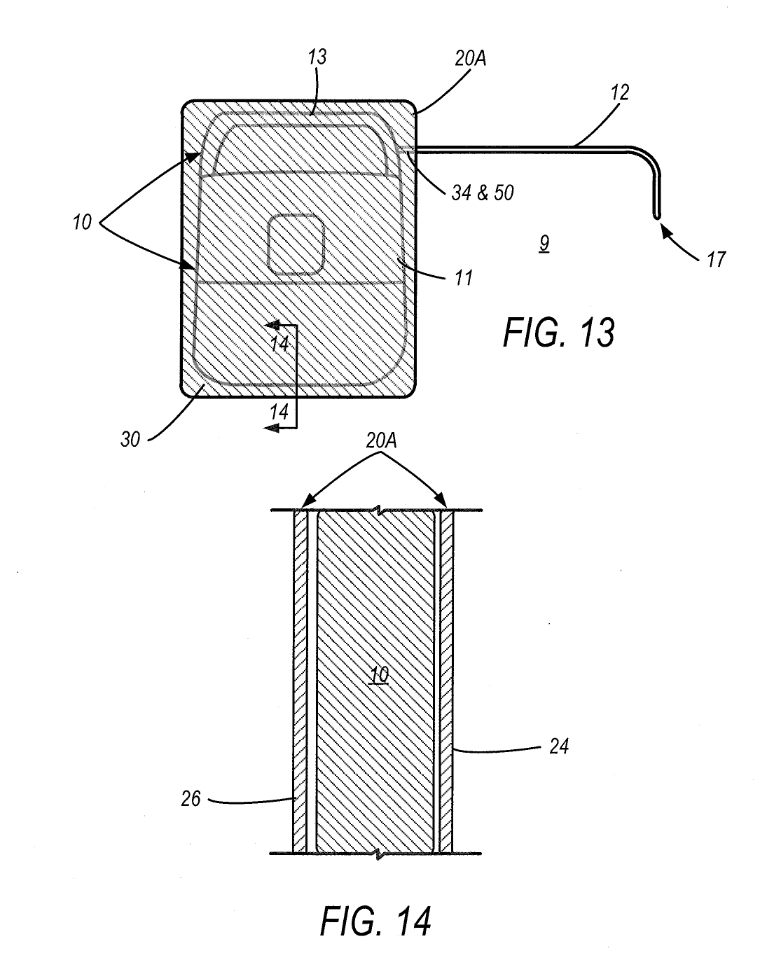

FIGS. 13 and 14 illustrate the trial SCS neurostimulation device 10 of FIG. 1 contained in an inner enclosure 20A of a combined enclosure 20 as will now be discussed. As shown in FIGS. 13 and 14, the device 10 and inner enclosure 20A are in readiness for affixing to the patient skin 9 surface via an outer enclosure 20B of the combined enclosure 20. Specifically, the lead 12, which has been implanted in the patient 9, extends from the header 13 of the device 10 and the inner enclosure 20A into the patient 9 via a percutaneous opening 17 in the patient 9. The inner enclosure 20A includes a patient side 24 and an outward-facing side 26 opposite the patient side 24. The inner enclosure 20A may be in the form of a waterproof polyethylene bag that is sealable around the device 10 and, in some embodiments, may fit the device 10 “like a glove” or be otherwise a tightly conform fit around the device 10. The inner enclosure 20A may be molded of soft flexible silicone rubber, polyethylene, polyurethane, polyurethane silicone rubber copolymer, butyl rubber, vinyl, latex, PET or similar pliable medical grade materials.

In some embodiments, the inner enclosure 20A may have a zip-lock style opening that allows the device 10 to be inserted into or removed from the inner enclosure 20A. In some embodiments, the inner enclosure 20A may be taped closed with a waterproof tape and enhanced with waterproof tape around the lead entrance inner enclosure 20A. In some embodiments, the inner enclosure 20A may be alternatively configured such that it is in the form of waterproof adhesive layers between which the device 10 is sealed, similar to the waterproof adhesive layers discussed above with respect to FIGS. 10 and 12.

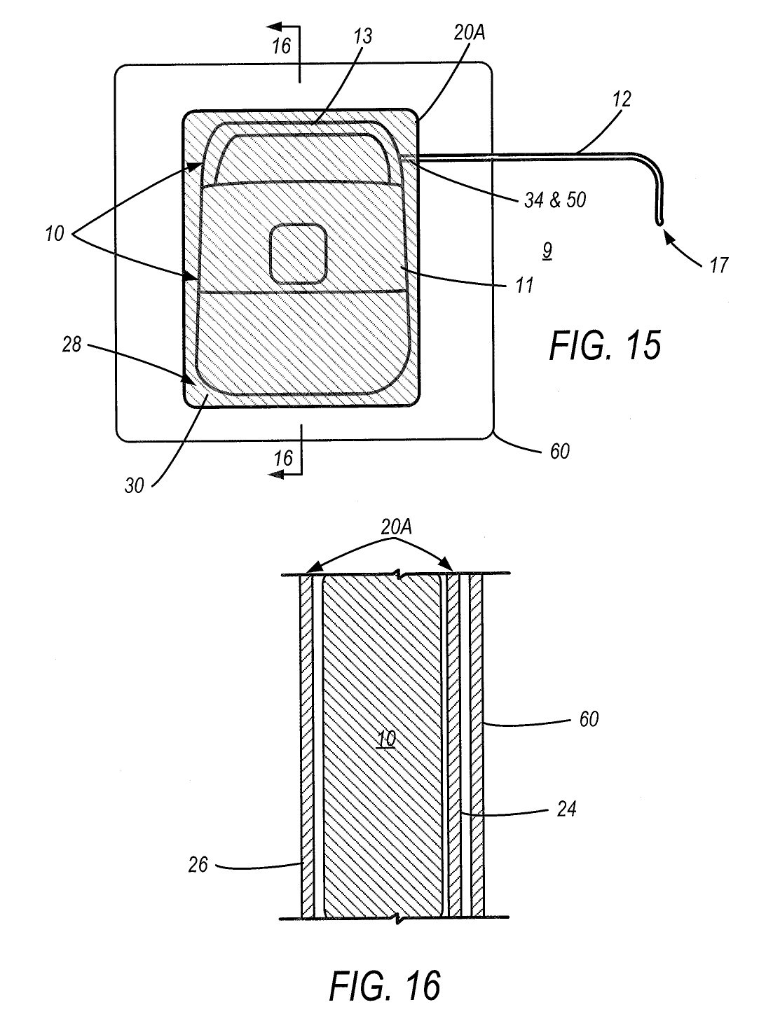

As shown in FIGS. 15 and 16, a bottom layer 60 of the outer enclosure 20B has been laid down on the patient skin surface 9 between the patient side 24 of the inner enclosure 20A and the skin surface 9. In one embodiment, the bottom layer 60 of the outer enclosure 20B may be a waterproof medical adhesive tape 60 or other waterproof material that is adhered to the patient skin surface. The tape 60 not only tolerates the presence of water but does not allow water to diffuse through the tape 60. In other embodiments, the bottom layer 60 of the outer enclosure 20B may be alternatively in the form of a breathable adhesive fabric layer 60. The bottom layer 60 of the outer enclosure 20B is sufficiently larger in area than the device 10 and inner enclosure 20A to isolate the device 10 and inner enclosure 20A from the patient skin surface 9.

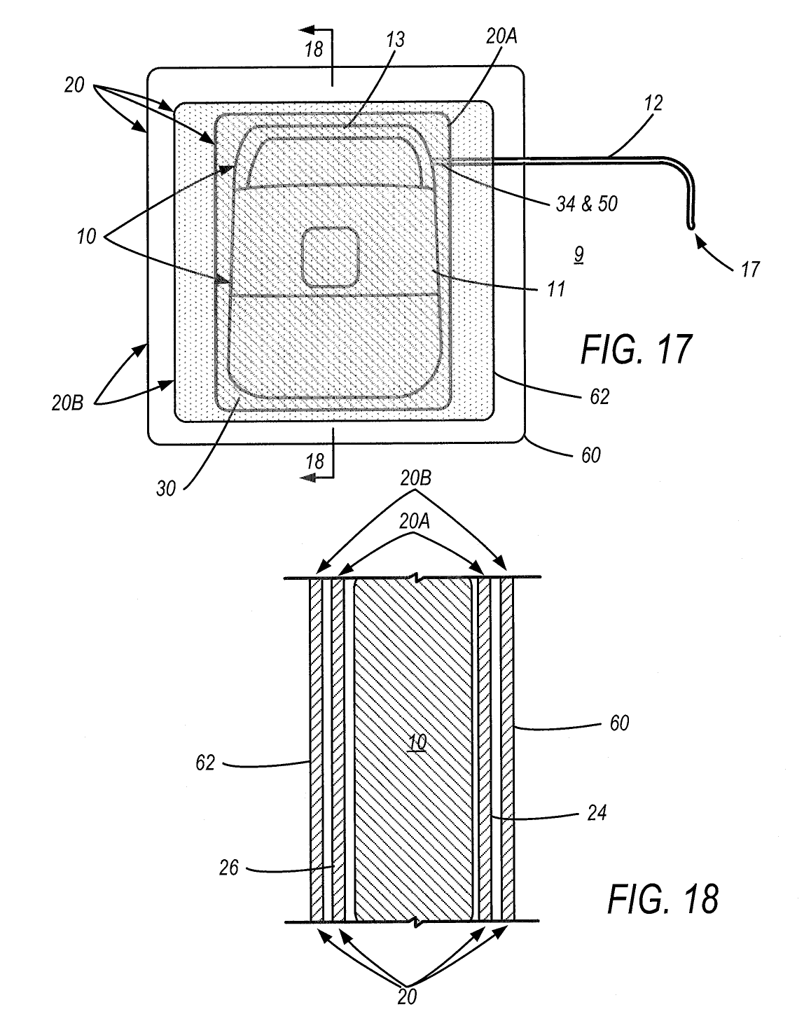

As illustrated in FIGS. 17 and 18, the outer enclosure 20B is completed by applying a top layer 62 of the outer enclosure 20B over outward-facing side 26 of the inner enclosure 20A and the bottom layer 60. In one embodiment, the top layer 62 of the outer enclosure 20B may be a waterproof medical adhesive tape 62 or other waterproof material that is adhered to the outward-facing side 26 of the inner enclosure 20A and the bottom layer 60 to sandwich the inner enclosure 20A (and the device 10 contained within the inner enclosure 20A) between the two layers 60, 62 of the outer enclosure 20B. In other embodiments, the top layer 62 of the outer enclosure may be alternatively in the form of a breathable adhesive fabric layer 60 that is not waterproof. The top layer 62 is sufficiently larger in area than the inner enclosure 20A but, in some embodiments, may be smaller in area than the bottom layer 60.

As can be understood from FIGS. 17 and 18, the device 10 is fully contained within the inner enclosure 20A and the outer enclosure 20B, which together form a combined enclosure 20. As can be understood from the preceding discussion, in some embodiments the inner enclosure 20A and outer enclosure 20B are both waterproof. In such an embodiment, both the inner and outer enclosures 20A, 20B act to prevent water from the patient showering from reaching the device 10 and also isolates the device 10 from contamination via other means.

In one embodiment, one or both layers 60, 62 of the outer enclosure 20B of FIGS. 17 and 18 are made from a micro-porous tape made of a hydrophobic material. Such a material can pass water vapor and thus provide a more comfortable breathable barrier as long as the pores are in the micron range to prevent liquid water or contaminants from passing through the layers 60, 62.

Also, as can be understood from the preceding discussion regarding FIGS. 17 and 18, in some embodiments the inner enclosure 20A will be waterproof while the outer enclosure 20B a breathable fabric that is not waterproof. In such an embodiment, only the inner enclosure 20A acts to prevent water from the patient showering from reaching the device 10. The outer enclosure 20B provides a breathable mechanism for attaching the device 10 to the patient 9, thereby increasing patient comfort. Both the inner and outer enclosures 20A, 20B work together as a combined enclosure 20 to isolate the device 10 from contamination via means other than water.

Following use, the bottom layer 60 is peeled off the patient 9 along with the trial SCS device 10, the inner enclosure 20A and the rest of the outer enclosure 20B. Then the two layers 60, 62 of the outer enclosure 20B are removed from about the inner enclosure 20A enclosing the device 10. The inner enclosure 20A is then cut off of or otherwise removed from about the device 10. Finally, the header 13 is discarded because it is contaminated with body fluids that end up on the lead 13 due to the lead implantation process. New batteries are inserted in the can 11 of the device 10 and the can 11 may be reused for the next patient.

It should be noted that the combined enclosure 20 of FIGS. 17 and 18 prevents the can 11 of the device 10 from coming into contact with the patient skin surface 9. Further, the combined enclosure 20 prevents the can 11 from getting contaminated with the body fluids that contaminate the lead 12.

FIG. 19 illustrates a connector assembly 100 for receiving and connecting with a lead connector end 50 of a lead 12, the connector assembly 100 being configured for placement in a header region 101 of a clamshell protective enclosure 20 as described below. The connector assembly 100 includes a printed circuit board 102 supporting slotted tubes 104, setscrew assemblies 106, and a mini HDMI connector 108. The connector assembly 100 may be the header of the trial SCS device 10 or the connector assembly 100 may be part of the header of the trial SCS device 10.

As can be understood from FIG. 20, each slotted tube 104 includes a series of slots 110 extending transversely into the side of the tube 104. Each tube 104 may be formed of an electrically insulating material such as, for example, plastic. Each tube 104 acts as a lead connector receptacle 32 of the header of the trial can 11, as explained below.

As shown in FIG. 21, a leaf spring connector 112 biases through each slot 110 into the interior cylindrical confines 114 of the tube 104. The leaf spring connectors 112 are formed of an electrically conductive metal and soldered to the printed circuit board 102, which serves as a substrate for the components of the connector assembly 100. The interior cylindrical confines 114 of the tube 104 has a diameter that is configured to accept the cylindrically configured lead connector end 50 of at a proximal end of the lead 12.

As can be understood from FIGS. 19 and 20, round holes 116 extend through each tube 104 near an entrance 118 of the tube 104. A setscrew assembly 106 is located at each such round hole 116, a setscrew 120 of such assembly 106 being aligned so as to be threadable within the round hole 116. Thus, the setscrew 120 can be used to secure the lead connector end 50 within the interior cylindrical confines 114 of the tube 104. An O-ring 122 is located at the entrance 118 of each tube 104 to provide a liquid seal about the lead connector end 50 when received in the interior cylindrical confines 114 of the tube 104.

As can be understood from FIG. 19, conductive traces 126 extend from respective leaf spring connectors 112 to electrically couple with the appropriate electrical aspects of the mini HDMI connector 108. Accordingly, when a lead connector end 50 is received in the interior cylindrical confines 114 of the tube 104, the leaf spring connectors 112 of the tube 104 bias against corresponding electrically conductive contact rings of the lead 12 to place the electrodes 14 of the lead 12 in electrical communication with the mini HDMI connector 108.

FIGS. 22 and 23 respectively illustrate an exterior surface 150 and an interior surface 152 of an outward half 62 of a clamshell protective enclosure 20 for employment with the connector assembly 100 of FIG. 19. Similarly, FIGS. 24 and 25 respectively illustrate an exterior surface 154 and an interior surface 156 of an inward half 60 of the clamshell protective enclosure 20. Each interior surface 152, 156 of the two enclosure halves 60, 62 is divided by interior walls 160 into an upper or header region 162 and a lower or can region 164. Each header region 162 includes openings 166 that daylight at the edges of the two enclosure halves 60, 62 to define at least a portion of the lead connector receptacles 32 that combine with the interior cylindrical confines 114 of the above-described tubes 104. Setscrew access holes 170 are defined in the outward half 62 of the clamshell protective enclosure 20 near the openings 166. The holes 170 are configured to receive a septum 175 that covers the above-described setscrews 120 went the connector assembly 100 is received in the upper region 162 of the clamshell protective enclosure 20 as described below.

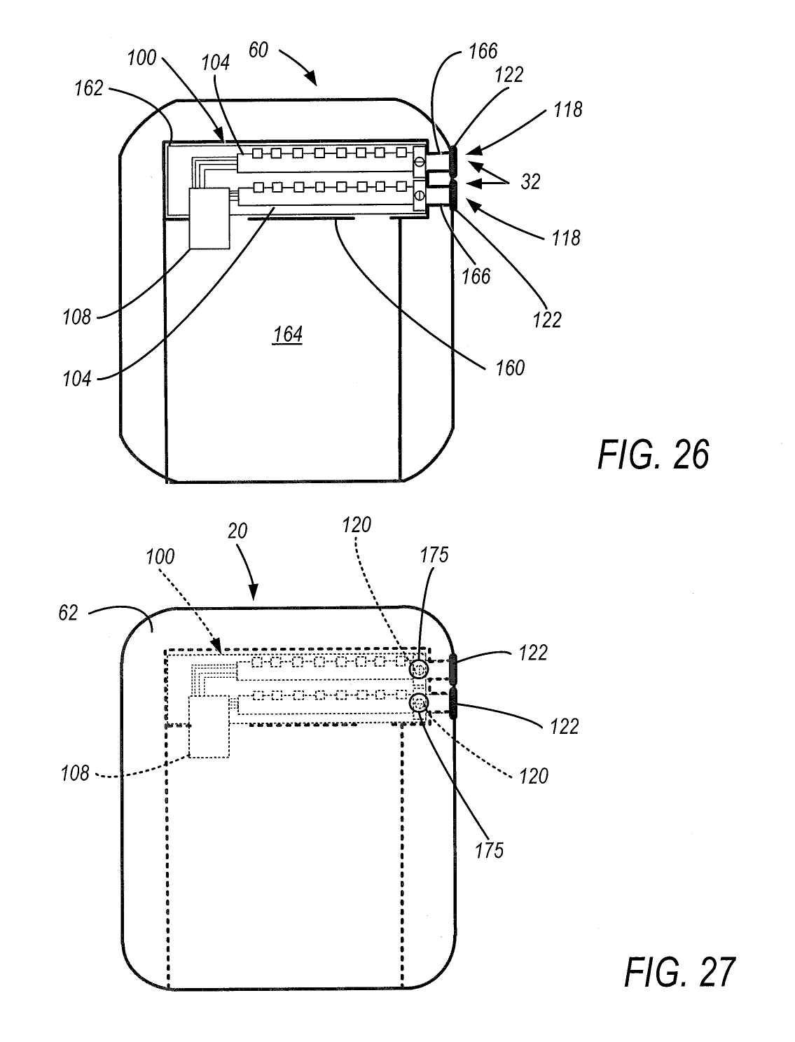

FIG. 26 depicts the connector assembly 100 of FIG. 19 occupying the upper region 162 of the interior 156 of the inward half 60 of the clamshell protective enclosure 20. As can be understood from FIG. 26, the mini HDMI connector 108 projects from the upper region 162 into the lower region 164. Also, the tubes 104 extend through the openings 166, and the O-rings 122 seal the openings 166 that define the lead connector receptacles 32. The O-rings 122 also seal around the leads 12 when received in the lead connector receptacles. The upper region 162 has features that allow the connector assembly 100 to snap-in place in the upper region 162.

FIG. 27 is the same view as FIG. 26, except the outward half 62 of the clamshell protective enclosure 20 has been mated with the inward half 60 to form the entirety of the clamshell protective enclosure 20 and enclose the connector assembly 110 therein. The two halves 60, 62 of the enclosure 20 can be bonded together with waterproof adhesive. Septums 175 occupy the holes 170 of the outward half 62 of the clamshell protective enclosure 20 and extend over the setscrews 120 to seal the holes 170 against fluid infiltration.

FIG. 28 is the same view as FIG. 27, except the lead connector ends 50 are received in the connector assembly 100 and the trial pulse generator portion or can 11 is in the process of being inserted into the clamshell protective enclosure 20. The can 11 includes a mini HDMI receptacle 200 that is configured to mechanically and electrically couple with the mini HDMI connector 108 projecting from the connector assembly 100. The clamshell protective enclosure 20 is fully assembled by the combination of the two halves 60, 62 as described with respect to FIG. 27. The leads 12 are implanted into the patient 9 such that the leads 12 extend into the patient 9 via percutaneous penetrations 17. The lead connector ends 50 extend into the lead connector receptacles 32, which are part of and positionally correspond with openings 34 defined in the enclosure 20 via the combination of the openings 166 of each of the two halves 60, 62 of the enclosure 20. A screwdriver or wrench passes through the septums 175 to tighten the setscrews 120 so as to secure the lead connector ends 50 in the connector assembly 100. As indicated by arrow B, the trial can 11 is inserted into the volume of the lower region 164 via an opening 210 that daylights the volume of the lower region 164 at the bottom of the enclosure 20.

FIG. 29 is the same view as FIG. 28, except the trial can 11 is fully located within the lower region 164 of the clamshell protective enclosure 20. The mini HDMI connector 108 is received in the mini HDMI receptacle 200, thereby coupling the trial can 11 with the connector assembly 100, which acts as the header for the trial can 11 and the overall trial SCS device 10 resulting from the joined combination of the can 11 and connector assembly 100. Once the trial can 11 is fully located within volume of the lower region 164, the opening 210 through which the can 11 passed to enter the volume of the lower region 164 can be waterproof sealed via a cover or waterproof tape that fills or extends over the opening 210.

As indicated in FIGS. 28 and 29, in one embodiment, the implanted leads 12 can be already coupled to the lead connector assembly 100 prior to the insertion of the trial can 11 into the enclosure 20. The enclosure 20, with the overall trial SCS device 10 contained therein, may then be taped to the patient 9.

Alternatively, the trial can 11 can be inserted into the enclosure 20 to create the overall trial SCS device 10 and then the lead connector ends 50 can be inserted into the lead connector assembly 100. The enclosure 20, with the overall trial SCS device 10 contained therein, may then be taped to the patient 9.

Following use, the cover or waterproof tape that fills or extends over the opening 210 is removed from the opening 210 and the trial can 11 is removed from within the enclosure 20. New batteries are inserted into the trial can 11 and the trial can 11 may be reused for the next patient. It should be noted that the trial can 11 does not come in contact with the patient's skin and does not get contaminated with the body fluids that contaminate the lead 12. The enclosure 20, along with the lead connector assembly 100 contained therein, is discarded because it is contaminated and designed to be very low cost so it is not worth cleaning or processing.

In general, while the invention has been described with reference to particular embodiments, modifications can be made thereto without departing from the scope of the invention. Note also that the term “including” as used herein is intended to be inclusive, i.e. “including but not limited to.”

What is claimed is:

1. A disposable enclosure for use with a trial neurostimulation device configured to electrically couple with a neurostimulation lead for implant within a patient, the trial neurostimulation device having a pulse generator portion, the disposable enclosure comprising:
a first wall structure;
a second wall structure opposite the first wall structure;
a volume between the first and second wall structures configured to receive therein the pulse generator portion; and
a header configured to electrically couple with the neurostimulation lead, the header supported in the disposable enclosure adjacent the volume and configured to electrically couple with the pulse generator portion when the pulse generator portion is located within the volume.

2. The disposable enclosure of claim 1, wherein the first and second wall structures are part of a bag-like structure.

3. The disposable enclosure of claim 1, wherein the at least one of the first wall structure or the second wall structure is formed of a pliable medical grade material.

4. The disposable enclosure of claim 1, wherein the at least one of the first wall structure or the second wall structure comprises at least one of silicone rubber, polyethylene, polyurethane, polyurethane silicone rubber copolymer, butyl rubber, vinyl, latex, or polyethylene terephthalate.

5. The disposable enclosure of claim 1, wherein the first and second wall structures are part of respective portions of a clam-shell configuration of the disposable enclosure.

6. The disposable enclosure of claim 1, wherein the header is supported in the disposable enclosure by being fixedly installed into the disposable enclosure as part of the manufacturing of the disposable enclosure.

7. The disposable enclosure of claim 6, wherein the header is molded into material extending from at least one of the first or second wall structures.

8. The disposable enclosure of claim 6, wherein the header is snap-fit into the disposable enclosure.

9. The disposable enclosure of claim 1, further comprising an opening extending into the volume and by which the pulse generator portion is inserted into the volume.

10. The disposable enclosure of claim 9, further comprising a lid configured to close off the opening.

11. The disposable enclosure of claim 1, wherein the header is a lead connector assembly comprising a mini HDMI connector, the pulse generator portion electrically coupling with the header via the mini HDMI connector.

12. The disposable enclosure of claim 12, wherein the mini HDMI connector projects into the volume.

13. The disposable enclosure of claim 1, wherein the header comprises a lead connector assembly comprising a mini HDMI connector, the pulse generator portion electrically coupling with the header via the mini HDMI connector.

14. The disposable enclosure of claim 13, wherein the mini HDMI connector projects into the volume.

15. A method of inhibiting contamination of a pulse generator portion of a trial neurostimulation device that is configured to electrically couple with a neurostimulation lead for implant within a patient, the method comprising:
enclosing the pulse generator portion in a volume of a disposable enclosure comprising a first wall and a second wall opposite the first wall, the volume being located between the first and second walls; and
attaching the enclosed pulse generator portion to the patient, the neurostimulation lead implanted in the patient and extending through the enclosure and electrically coupled with the trial neurostimulation device.

16. The method of claim 15, wherein the first wall is laid down on and adhered to a skin surface of the patient, the pulse generator portion is placed over the first wall, and the second wall is laid down over both the pulse generator portion and the first wall, the second wall being adhered to the first wall.

17. The method of claim 16, wherein at least one of the first or second walls comprise a waterproof medical adhesive tape.

18. The method of claim 16, wherein the second wall covers a smaller area than the first wall.

19. The method of claim 16, wherein the pulse generator portion is sealed between the first and second walls.

20. The method of claim 15, wherein the disposable enclosure is an inner disposable enclosure, the method further comprising enclosing the inner disposable enclosure within an outer disposable enclosure.

21. The method of claim 20, wherein the outer disposable enclosure comprises a third wall and a fourth wall opposite the third wall, a volume of the outer disposable enclosure being defined between the third and fourth walls, the enclosed pulse generator portion being located in the volume of the outer disposable enclosure.

22. The method of claim 21, wherein the third wall is laid down on and adhered to a skin surface of the patient, the pulse generator portion enclosed in the inner disposable enclosure is placed over the third wall, and the fourth wall is laid down over both the pulse generator portion that is enclosed by the inner disposable enclosure and the third wall, the fourth wall being adhered to the third wall.
